(19) United States
(12) Patent Application Publication
(10) Pub. No.: US 2007/0231334 A1
(43) Pub. Date: Oct. 4, 2007

(54) Combination therapy for anthrax using antibiotics and protease inhibitors

(76) Inventors: Ken Alibek, Fairfax, VA (US); Taissia Popova, Bristow, VA (US); Serguei Popov, Bristow, VA (US); Svetlana Hopkins, Arlington, VA (US)

Correspondence Address: HOGAN & HARTSON L.L.P., 1999 AVENUE OF THE STARS, SUITE 1400, LOS ANGELES, CA, 90067, US

(21) Appl. No.: 11/231,600

(22) Filed: Sep. 21, 2005

Related U.S. Application Data
(63) Continuation-in-part of application No. 11/041,881, filed on Jan. 25, 2005.
(60) Provisional application No. 60/612,616, filed on Sep. 24, 2004.
(60) Provisional application No. 60/615,591, filed on Oct. 5, 2004.
(60) Provisional application No. 60/622,112, filed on Oct. 27, 2004.

(51) Int. Cl. A61K 39/40; A61K 31/195; A61K 31/473; A61K 36/48; A61K 31/366

(52) U.S. Cl. 424/164.1; 514/566; 424/757; 514/298; 514/460

(57) ABSTRACT

The invention provides compositions for treatment anthrax infection. The composition comprises a therapeutically effective amount of at least one B. anthracis metalloprotease inhibitor. The composition may further include an antimicrobial agent. The invention also provides methods for treating anthrax infection in a human or an animal subject. The method comprises administering to the subject a therapeutically effective amount of a composition of the present invention.

This application is a continuation-in-part of the U.S. patent application Ser. No. 11/041,881, filed Jan. 25, 2005, which claims priority under 35 U.S.C. § 119(e) to U.S. provisional application No. 60/612,616, filed Sep. 24, 2004, U.S. provisional application No. 60/615,591, filed Oct. 5, 2004, and U.S. Provisional application No. 60/622,112, filed Oct. 27, 2004, all of which are incorporated herein by reference. This application also claims the benefit of U.S. provisional application No. 60/612,616, filed Sep. 24, 2004 and U.S. provisional application No. 60/615,591, filed Oct. 5, 2004.

This invention was made with partial Government support under contract numbers W9111NF-04-C-0046 and MDA972-02-C-0067 awarded by Defense Advanced Research Project Agency (DARPA). The Government has certain rights in the invention.

### BACKGROUND OF THE INVENTION

This invention relates to compositions and methods of treating anthrax.

Anthrax is a severe, often fatal disease caused by systematic spread of sporulating bacteria Bacillus anthracis (B. anthracis). High mortality rates in anthrax patients is often attributed to a combination of causes, including profound hemorrhagic syndrome, disruption of the respiratory system function (due to pleural effusion, atelectasis in the lungs, accumulation of mucous in the alveoli and bronchioles, increased permeability, and vasculitis in the lung vessels), and shock. The hemorrhages, which are seen in 100% of the cases of inhalational anthrax, are often (50-70% of the time) complicated by severe meningitis, leptomeningitis, or hematomas in the brain tissue [Alibek et al, 2004]. Thus, if used as a biological weapon, B. anthracis is expected to cause massive casualties and high mortality rate.

The anthrax toxin has been determined to be the primary virulence factor in anthrax infection and mechanisms of its toxicity are well documented [Popov et al., 2002; Moayeri, 2004]. The anthrax toxin is composed of three factors, protective antigen [PA], lethal factor [LF], and edema factor [EF]. A combination of PA and EF produces Edema toxin [EdTx] and a combination of PA and LF forms Lethal toxin [LeTx].

Current anthrax therapies focus on inhibiting activity of anthrax lethal toxin. While this approach has provided some positive outcomes, the existing therapies are not highly effective. There is, therefore, a compelling need to develop new more effective compositions and methods for treatment of anthrax infections.

### BRIEF SUMMARY OF THE INVENTION

The invention fulfills this need in the art by providing treatments for anthrax. In one aspect, the invention provides a composition for treating an anthrax infection comprising a therapeutically effective amount of at least one B. anthracis metalloprotease (MP) inhibitor, wherein the MP is other than LF. The MP inhibitor may be a chemical inhibitor, including, but not limiting to, ethylenediamine-tetraacetic acid (EDTA), phosphoramidon, soybean trypsin inhibitor (SBTI), o-phenanthroline, aprotinin, galardin, disulfram, and ebelactone B. The MP inhibitor may also be an antibody raised against a MP. In one embodiment, the antibody is raised against at least one peptide comprising a sequence SEQ ID NO:1, HEFTHYLQGRYEVPGL; SEQ ID NO:2, DVIGHELTHAVTE; SEQ ID NO:3, ADYTRGQGIETY, or a conservative modification of any of these sequences.

In another aspect, the invention provides a composition for treating an anthrax infection comprising a therapeutically effective ratio of at least one B. anthracis MP inhibitor and an antimicrobial agent. In one embodiment, the antimicrobial agent is an antibiotic. The antibiotic may be a fluoroqinalone, tetracycline, β lactam, or another antibiotic effective against anthrax infection. In one embodiment, the antibiotic is ciprofloxacin or doxycycline.

In still another aspect, the present invention provides methods for treating anthrax infection in a human or an animal subject. In one embodiment, a method comprises administering to the subject a therapeutically effective amount of a composition comprising at least one B. anthracis MP inhibitor. In another embodiment, a method comprises administering to the subject a composition comprising a therapeutically effective ratio of at least one B. anthracis MP inhibitor and an antimicrobial agent. In methods of the present invention, the administering step may be delayed at least 24 hours from the time of exposure of the subject to B. anthracis.

### BRIEF DESCRIPTION OF THE DRAWINGS

The above-mentioned and other features of this invention and the manner of obtaining them will become more apparent, and will be best understood by reference to the following description, taken in conjunction with the accompanying drawings. These drawings depict only typical embodiments of the invention and do not therefore limit its scope.

FIG. 1A depicts an SDS-PAGE gel of B. anthracis culture supernatant (“BACS”) fractions separated on a size exclusion column. Western blots using specific antisera are depicted in FIGS. 1B, 1C, and 1F as follows: FIG. 1B: a-M4EL; FIG. 1C, left panel: a-M4AC; FIG. 1C, right panel: a-M4EP; FIG. 1F: a-M9Coll. Zymograms of caseinolytic and collagenolytic activities of BACS are depicted in FIGS. 1D and 1E, respectively. The molecular masses (KDa) of the marker proteins are indicated by arrows. In FIG. 1A, s denoted BACS, and numbers above correspond to column fractions. In FIG. 1E, different amounts of BACS were loaded on a gel (15 μl, 7 μl and 3 μl, from left to right).

FIG. 2a depicts hemorrhagic activity of culture supernatants. This activity is depicted in graphic representation in FIG. 2b. FIG. 2c shows the effect of chemical inhibitors on hemorrhagic activity. FIG. 2d shows hemorrhagic reaction induced by subcutaneous infection of secreted proteins of B. anthracis (delta-pX01/pX02 strain). Panel A of FIG. 2d depicts hemorrhagic activity of 30 μg secreted proteins of B. anthracis. Panel B of FIG. 2d is a control LeTx (100 PA and 100 μg LF). FIG. 2e demonstrates hemorrhagic reaction induced by infra-arterial infusion towards brain of secreted proteases of B. anthracis (delta-pX01/pX02 strain) and LeTx. In panel A of FIG. 2e, 135 μg secreted proteins of B. anthracis were applied. Panel B of FIG. 2e is control with LeTx (100 μg PA and 100 μg LF).

FIG. 3A depicts the survival of mice upon intratracheal injection of BACS. FIG. 3B depicts the survival of mice infused with secreted proteins of B. anthracis or with LeTx or PBS. Each animal was infused with 50 μl of volume and observed for mortality. Each group contained five animals.

FIG. 4 depicts protection of mice against B. anthracis (Sterne) infection by administration of ciprofloxacin.

FIG. 5A depicts protection of mice against B. anthracis (Sterne) by administration of ciprofloxacin in combination with phosphoramidon for 10 days beginning at 24 hours and 48 hours post spore challenge. FIGS. 5B and 5C depict protection of mice against B. anthracis (Sterne) infection by administration of ciprofloxacin or ciprofloxacin in combination with 1,10-phenanthroline (o-phenanthroline) for 10 days, beginning 24 hours (5B) and 48 hours (5C) post spore challenge.

FIGS. 6A, B, and C depict post-exposure efficacy of hyperimmune rabbit sera in mice challenged with B. anthracis (Sterne). Treatment with sera alone (FIG. 6A) or in combination with ciprofloxacin (FIGS. 6B and C) was initiated 24 hours post exposure and continued for 10 days once daily.

FIGS. 7A, B, C, and D depict protection of mice against B. anthracis (Sterne) infection by administration of ciprofloxacin or doxycycline alone or in a combination with chemical inhibitors for 10 days beginning 48 hours post spore challenge. FIG. 1A depicts the effects of aprotinin; FIG. 1B depicts the effects of galardin; FIG. 1C depicts the effects of disulfuram; FIG. 1D depicts the effects of ebelactone B.

### DETAILED DESCRIPTION OF THE PREFERRED EMBODIMENTS

Lethal toxin, which is secreted by proliferating B. anthracis, is one of the factors that is widely believed to be a major cause of death in human and in several susceptible animal species (Inglesby et al., 2002). It has been suggested, for example, that the lethal action of anthrax toxin may be inactivated by molecules that inhibit the protease activity of LF (Panchal et al., 2004). However, the pathology observed in experimental animals exposed to lethal toxin is drastically different from that found during the natural infectious process. In fact, recent extensive analyses in mice and rats challenged with a highly purified lethal toxin (Moayeri et al, 2003; Chui et al., 2004) confirm earlier observations (Klein et al., 1996) that toxin activity causes no gross pathology, such as hemorrhagic syndrome, profound vasculitis, effusion in the thorax, and severe respiratory syndrome, but only manifests in hypoxic liver failure.

Furthermore, in the cadavers of people and animals who have died of anthrax, the process of cadaver decay takes just 12-24 hours, instead of the usual 2-3 days. In addition, the cadavers exhibit green and purple livores mortis on external examination, enormous abdominal distension, and nearly absent postmortem rigidity. Again, these changes indicate that previously unknown virulence mechanisms may be at work.

Finally, the inventors discovered that proteins secreted by delta Sterne strain of B. anthracis, which is devoid of both toxigenic plasmids and produces neither lethal nor edema toxins, are directly lethal to mice upon intra-tracheal administration at doses as low as 10 μg per mouse. In fact, the death of animals exposed to these proteins can take place as fast as only a few hours after administration of the proteins. Therefore, the pathogenesis of anthrax is not due solely, or even at all, to LeTx or EdTx.

Generally, the capacity of bacteria to cause destruction of tissues, degradation of immunoglobulins and cytokines, the release of inflammatory mediators or the activation of host proteolytic enzymes, is attributed to a wide variety of secreted proteolytic enzymes (also referred to as proteases) (Supuran et al., 2002). The present invention is based on a discovery that certain metalloproteases, other than LF, are responsible for the unexplained pathology of anthrax infections.

To identify B. anthracis proteases with the highest virulence-enhancing activity, genomes of two virulent anthrax strains (Read et al., 2002; Read et al., 2003) and two avirulent species from the same family, B. cereus (Ivanova et al., 2003) and B. subtilis (Kunst et al., 1997), were compared with the known sequence motifs of hundreds of families of proteolytic enzymes. As discussed in more detail in Example 2, metallo-protease (MP) enzymes, including, but not limited to M4 family of thermolysin/elastase-like neutral proteases and the M9 family of collagenases, were identified as the candidate virulence-enhancing factors of B. anthracis.

Accordingly, in its first aspect, the present invention provides a composition for treating an anthrax infection. The composition comprises a therapeutically effective amount of a B. anthracis MP inhibitor, wherein MP is other than LF. In one embodiment, the MP is selected from the group consisting of proteases that are members of M4 family of thermolysin and elastase-like neutral proteases and proteases that are members of M9 family of collagenases.

The inventors also discovered that secreted MPs of B. anthracis can digest protein substrates, such as casein and gelatin in vitro (Example 5), and can induce a hemorrhagic process in test subjects in vivo (Example 3). The inventors further discovered that these activities are inhibited by inhibitors of MPs, including, but not limited to chemical inhibitors (Examples 3, 7, and 9) and antibodies raised against MPs (Examples 4 and 8).

Accordingly, in one embodiment of the present invention, a compound that inhibits activity of a metalloprotease of B. anthracis is a chemical inhibitor. Such chemical inhibitors, include, but are not limited to, ethylenediaminetetraacetic acid (EDTA), phosphoramidon, soybean trypsin inhibitor (SBTI), o-phenanthroline, aprotinin, galardin, disulfram, or ebelactone B.

Aprotinin is a basic single-chain polypeptide that inhibits serine proteases by binding to the active site of the enzyme and forming a tight complex. It inhibits plasmin, kallikrien, trypsin, chymotrypsin, and urokinase. It does not inhibit carboxypeptidase A and B, papain, pepsin, subtillisin, thrombin, and factor X. It is used in cell culture to prevent proteolytic damage to cells and extend the lifetime of cells. The trade name for aprotinin is TRASYLOL® (Bayer Pharmaceuticals Corporation, West Haven, Conn.) and it has been approved by the FDA for certain cardiovascular disorders in 1998. The drug is now used to reduce blood loss following surgery or transplant and has been administered in the treatment of acute pancreatitis. It is a potent inhibitor of thermolysin and other bacterial metallo-endopeptidases. Aprotinin may be administered intravenously with a test dose of 1.4 mg, and a loading dose of 140-280 mg. Administration may be continued at 10-100 mg/hour, 25-50 mg/hour, or 35-70 mg/hour.

Disulfuram (Tetraethylthiuram disulfide) possesses antiretroviral activity, and has type IV collagenase inhibitory activity, which can be responsible for blocking invasion and angiogenesis through cell-mediated and non-cell mediated pathways. It is an FDA-approved drug for management of alcoholism, which is sold under the brand name ANTABUSE® (Ayerst, N.J.). Disulfuram may be administered orally at doses up to 500 mg/day. In one embodiment, the dose is 125-500 mg/day.

Galardin (also known as GM6001 (Glycomed Inc., Alameda, Calif.) or Ilomastat) is a metallopoteinase inhibitor of P. aeruginosa elastase, P. mirabilis proteinase, and E. faecalis gelatinase. It also inhibits human metalloproteases 1 (fibroblast collagenase), 2 (gelatinase), 3 (stromelysin), 8 (neutrophil collagenase), and 9 (gelatinase). It is widely used in cancer clinical studies. Currently, galardin is in development for treatment of inflammatory respiratory diseases such as smoking-related emphysema and COPD. It was previously under development (Phase 2) by Glycomed (Ligand) for opthalmological indications as an angiogenesis inhibitor, but development was discontinued. The drug is also used for treatment of corneal cancer, corneal ulcers, and scars (see, for example, U.S. Pat. No. 6,379,667, relevant parts of which are incorporated herein by the reference).

A single dose of galardin may be delivered to the middle ear at a dose from about 0.1 mg to about 50 mg. In some embodiments, a single dose of galardin is from about 1 mg to about 20 mg. In other embodiments, the dose is about 5 mg. If the galardin is delivered, for example, in the form of a liquid, a dose may be 100 microliters of a 50 mg/ml solution of galardin in a suitable liquid carrier. Dose frequency may be from once daily to six times daily. Alternatively, sustained continuous release formulations of galardin may be appropriate. Various formulations and devices for achieving sustained release are known in the art. In one embodiment, dosages for galardin may be determined empirically in individuals who have been given one or more administration(s) of galardin based on results of the initial administration(s). The galardin formulation may be administered for a duration of up to one year depending on the indication. Higher or lower doses may be used at the discretion of the clinician, as well as greater or lesser frequency of application.

Ebelactone B, o-phenathroline, posphoramidon, and soybean trypsin inhibitor may be administered invtravenously, parenterally, or as oral gavage, in a concentration of about 1 mg/kg or greater. In one embodiment, the inhibitor is administered in a concentration from about 1 mg/kg to about 4 mg/kg. Inventors further discovered that post-exposure administration of antibodies raised against B. anthracis MPs, such as MPs of M4 or M9 family, provided a substantial protective effect to mice challenged with B. anthracis (Examples 4 and 8). Accordingly, in another embodiment, B. anthracis metalloprotease inhibitor of the present invention comprises an antibody raised against peptides representing the common motifs of several B. anthracis MPs, including, but not limited to SEQ ID NO:1, HEFTHYLQGRYEVPGL; SEQ ID NO:2, DVIGHELTHAVTE; SEQ ID NO:3, ADYTRGQGIETY, or a conservative modification of any of these sequences.

The antibodies of the invention may be polyclonal or monoclonal. Monoclonal antibodies may be prepared as described by Kohler and Milstein (1975). Monoclonal antibodies may be engineered to be chimeric antibodies, including human constant regions. The antibodies of the invention may be raised in any species of animal, including but not limited to, rabbits, sheep, horses, mice, goats, monkeys, rats, etc. In one embodiment, antibodies are raised in a sheep.

For the purposes of the present invention, the term “conservative modification” refers to a change in the amino acid composition of a peptide that does not substantially alter its activity. Such conservative modifications are known to those skilled in the art and may include substitutions, deletions or additions which alter, add or delete a single amino acid or a small percentage of amino acids, e.g., often less than 5%, in the amino acid sequence.

For example, conservative modification may comprise of substitution of amino acids with other amino acids having similar properties such that the substitutions of even critical amino acids does not substantially alter activity. Conservative substitution tables providing functionally similar amino acids are well known in the art. The following six groups each contain amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W) (see also, Creighton, 1984, Proteins, W. H. Freeman and Company).

A conserved modification may also include mutating the amino acid residues that are not surface exposed in the native protein. These residues should not interact with the antibody and so changing them should still produce an antibody of equivalent affinity. Another example of conservative modification is adding amino acids to the N or C terminus that are not existent in the native protein sequence, but would increase antibody production. Peptides obtained by such additions are often referred to as constructs.

In another embodiment, the MP inhibitor is an antiserum containing at least one antibody raised against at least one peptide comprising a sequence SEQ ID NO:1, HEFTHYLQGRYEVPGL; SEQ ID NO:2, DVIGHELTHAVTE; SEQ ID NO:3, ADYTRGQGIETY, or a conservative modification of any of these sequences.

A single antibody or a mixture of different antibodies may be administered to a patient. In one embodiment, a single antibody in the amount from about 100 mg to about 400 mg is administered alone or in a combination with one or more additional antibodies to a patient. In another embodiment, the amount of an antibody is from about 120 to about 360 mg. In another embodiment, the amount of an antibody is from about 180 to about 300 mg. In yet another embodiment, the amount of an antibody is from about 200 to about 280 mg.

The amount of an antibody to be administered can also be determined on a per weight basis. In the present invention, a dose of an antibody to be administered, alone or in a combination with one or more additional antibodies, to a patient may be in a range from about 0.1 mg/kg to about 100 mg/kg or more. Other embodiments include doses of about 1 mg/kg to about 50 mg/kg. In yet other embodiments the amount of antibody is from about mg/kg to about 25 mg/kg. In further embodiment, about 10 mg/kg of an antibody is administered to a patient.

It is further discovery of the inventors that a strong synergistic enhancement of survival rates (up to 90% protection) and a faster recovery rates are achieved when a post-exposure therapy combines compounds that inhibit B. anthracis proteases with antimicrobial agents. Accordingly, in another aspect, the present invention provides a composition for treating an anthrax infection comprising a therapeutically effective ratio of at least one B. anthracis MP inhibitor, such as one of the inhibitors described above, and an antimicrobial agent.

For the purposes of the present invention, the term “antimicrobial” is used generally to include any agent that is harmful to microbes, including agents with antibacterial, antifungal, antialgal, antiviral, antiprotozoan and other such activity. The term “antibiotic” is used in the present invention to refer to an antibacterial agent.

In one embodiment of the invention, the antimicrobial agent and the B. anthracis MP inhibitor are administered at the same time. In another embodiment, the antimicrobial agent and B. anthracis MP inhibitor are administered serially, with either the antimicrobial agent or the MP inhibitor administered first.

In one embodiment, B. anthracis MP inhibitor is an antibody raised against a MP. An antibody may be administered intravenously or subcutaneously, and antibiotics may be administered orally, intravenously, or subcutaneously. Injectable forms of the antibiotics or antibodies can be administered intravenously or subcutaneously, while oral administration can be achieved by many different methods, including but not limited to, tablets, solutions, lozenges, etc. In still another embodiment, B. anthracis MP inhibitor is a chemical inhibitor.

In one embodiment the antimicrobial agent is an antibiotic. Although a broad range of antibiotics may be co-administered with the B. anthracis MP inhibitor, in one embodiment, the antibiotic is one that is recommended for treatment of anthrax, including but not limited to fluoroqinalones, such as ciprofloxacin hydrochloride (also referred to as ciprofloxacin), tetracyclines, such as doxcycline, and β lactams. In one embodiment, the composition comprises ciprofloxacin and a B. anthracis MP inhibitor selected from a group consisting of o-phenanthroline, aprotinin, and galardin. In another embodiment, the composition comprises doxycycline and a B. anthracis MP inhibitor is disulfuram or galardin.

The antibiotic may be administered orally, subcutaneously, or intravenously. In one embodiment, ciprofloxacin is administered orally or intravenously. When ciprofloxacin is administered orally, a single dose of about 100 mg to about 750 mg may be administered every twelve hours. In one embodiment, the dose of ciprofloxacin is about 250 mg. In another embodiment, the dose of ciprofloxacin is about 500 mg. In still another embodiment, ciprofloxacin is administered to children orally in an amount of about 15 mg/kg per dose, up to about 500 mg per dose.

In another embodiment, ciprofloxacin is administered intravenously every twelve hours in doses ranging from about 200 to about 400 mg. In still another embodiment, ciprofloxacin is administered to children intravenously at an amount of about 10 mg/kg, up to about 400 mg per dose. Treatment with ciprofloxacin may last from 5 to 60 days.

In another embodiment, doxycycline is administered either orally or intravenously in an amount of from about 20 to about 750 mg every twelve hours. In one embodiment, doxycycline is administered in an amount selected from the group consisting of 20 mg, 50 mg, 100 mg, 200 mg, 250 mg, and 500 mg of doxycycline. In one embodiment, an initial dose of 200 mg is administered before a maintenance treatment. In children over eight years of age and weighing 100 lbs or less, the recommended dose is 2 mg/lb on the first day, divided into two doses, followed by 1 mg/lb as one dose or two on subsequent days. The maintenance dosage may also be give at 2 mg/lb.

In general, the frequency of administration of the compounds of the invention may be determined and adjusted over the course of therapy, and is generally, but not necessarily, based on treatment and/or suppression and/or amelioration and/or delay of symptoms and clinical findings.

The compositions of the invention may be incorporated into liposomes or may be micorencapsulated for administration to a patient. Other methods of stabilizing the compositions in the blood can also be used in the invention.

In another aspect, the present invention provides methods for treating anthrax infection in a human or an animal subject. In one embodiment, a method comprises administering to the subject a therapeutically effective amount of a composition comprising at least one B. anthracis MP inhibitor. In another embodiment, a method comprises administering to the subject a composition comprising a therapeutically effective ratio of at least one B. anthracis MP inhibitor and an antimicrobial agent. In methods of the present invention, the administering step may be delayed at least 24 hours from the time of exposure of the subject to B. anthracis.

For the purposes of promoting an understanding of the principles of the invention, reference will now be made to the embodiments illustrated in the drawings and described in the examples that follow. Specific language will be used to describe the same. It will nevertheless be understood that no limitation of the scope of the invention is thereby intended. Any alterations and further modifications in the described compositions and methods for treatment of anthrax infection, and any further applications of the principles of the invention as described herein are contemplated as would normally occur to one skilled in the art to which the invention relates.

### EXAMPLE 1

Materials and Methods

Microbial strains. The non-encapsulated Bacillus anthracis strain 34F2 (Sterne) [pXO1+, pXO2−] obtained from the Colorado Serum Company (Boulder, Colo.) was used in animal challenge experiments. The 50% lethal doses (LD₅₀s) by the inraperitoneal (i.p.) route were established earlier (Popov et al., 2004) and the LD₅₀ value for intraperitoneal challenge for DBA2 mice was found to be 3×10⁶ spores per mouse. The non-encapsulated, atoxigenic strain of B. anthracis (delta Ames) [pXO1−, pXO2−] was provided by Dr. J. Shiloach (National Institutes of Health, Bethesda, Md.). B. cereus strain ATCC #11778 and B. subtilis strain #23857 were purchased from American Type Culture Collection (Manassas, Va.).

Mice. Female DBA2 mice (9 weeks old) were obtained from Taconic (Germantown, N.Y.) and were used in all experiments described in the examples that follow.

Reagents. The following substances were used: ciprofloxacin (ICN Biomedicals, lot no. 4913F), phosphoramidon disodium salt, and 1,10-phenanthroline (Sigma, MO), EDTA (GibcoBRL), soybean trypsin inhibitor from Glycine max (Sigma, MO), thermolysin (EC 3.4.24.27) from Bacillus thermoproteolyticus (Sigma, MO). The fluorescently labeled casein and collagen type I for determination of proteolytic activity were from Molecular Probes (OR). Zymogram gels were from Invitrogen (Carlsbad, Calif.). Lethal factor (LF) and protective antigen (PA) were from List Biological Laboratories (CA).

Preparation of secreted proteins. Secreted substances were prepared by culturing B. anthracis (delta Ames) in LB media overnight. Cells were removed by centrifugation at 8000 g, and the supernatant was sterilized by filtration through 0.22 μm cellulose acetate filtration system (Corning, N.Y.) and further concentrated 50-fold using Amicon Ultra 15 centrifugal filter devices (10K cut-off pore size) (Millipore, MA). The proteins were used immediately after preparation or were stored at 4° C. for several days. Protein content was determined using Bradford reagent (Bio-Rad) with bovine serum albumin as standard. Slow reduction in the hemorrhagic activity was found upon storage within a week.

Fractionation of culture supernatants. 1 ml of B. anthracis culture supernatant (BACS) was loaded onto the size-exclusion Superdex® column (25×60, Pharmacia Biotech) and was eluted with PBS (pH 7.4) with a flow rate of 2 ml/min. Fractions of eluate were concentrated to equal volumes using Centricon® devices (Millipore, MA) with a 10K cut-off pore size.

Hemorrhages in femoral artery region. Mice were anesthetized by intraperitoneal injection of Avertin (2,2,2 tribromethanol, Aldrich) and 100 μl of B. anthracis secreted proteins (20 to 100 μg) were subcutaneously (sc) injected into the femoral artery region for observation of hemorrhagic changes after 3 to 15 hours. In order to record hemorrhagic changes animals were anesthetized by i.p. injection of Avertin and the fur over the femoral artery region was removed to allow open observation of a 1.5 to 2.5 cm² area of skin. It was photographed, and the size of the hemorrhagic spot was measured. In the experiments on the inhibition of hemorrhagic effect the secreted proteins were preincubated with specific antisera or protease inhibitors for 30 minutes on ice.

Generation of antibodies against B. anthracis MPs. The Invitrogen (CA) custom service was used to obtain rabbit polyclonal sera against the peptides listed in Table 1 conjugated with kallikrein. Two animals were immunized by each conjugate. All six rabbit sera had ELISA titers ranging from 100,000 to 200,000. For generation of murine polyclonal antibodies against the M4 protease (BA3442) the C-terminal part of the gene encoding amino acids 248 to 532 was cloned into pTrcHis2 TOPO TA cloning vector (Invitrogen, CA). The recombinant protein containing a 6×His tag was expressed in E. coli and purified using the Ni-NTA resin (Quiagen, CA). Mice were immunized with 50 μg of the protein emulsified in a complete Freund's adjuvant and were given two booster immunizations using an incomplete adjuvant with 2 week intervals. Serum was collected after two weeks since the last boost injection. In the skin hemorrhagic test described above, 30 μl of serum was able to completely suppress the hemorrhage caused by 30 μl of BACS.

TABLE 1
Sera against B. anthracis proteases

| Serum | Protein | | Gene | | |
|---|---|---|---|---|---|
| # | family | Protein | number | Antigen | Designation |
| 1 | M4 | Elastase- | BA3442 | Recombinant polypeptide | M4EL |
| | | like | | corresponding to the | |
| | | neutral | | fragment 248-532. | |
| | | protease | | | |
| 2 | M9 | Collagenase | BA0555, | HEFTHYLQGRYEVPGL (SEQ | M9Coll |
| | | | BA3299, | ID NO:1) spanning the | |
| | | | BA3584 | region of active center | |
| 3 | M4 | Neutral | BA5282, | DVIGHELTHAVTE (SEQ ID | M4AC |
| | | protease | BA0599 | NO:2) spanning the | |
| | | | | region of active center | |
| 4 | M4 | Neutral | BA2730 | ADYTRGQGIETY (SEQ ID | M4EP |
| | | protease | | NO:3) distant from the | |
| | | | | active center | |

Intratracheal delivery of B. anthracis secreted proteins. Mice were anesthetized by i.p. injection of Avertin and a 24 G angiogenic catheter (BD Biosciences, CA) was inserted into the trachea. 50 μl of experimental mixture, containing 10 to 100 μg of culture supernatant proteins were slowly injected through the catheter connected to a microsyringe. The angiogenic catheter was removed and animals were left for further observation. The untreated control group received the same volume of phosphate-buffered saline (PBS). A control group of three animals was injected with 50 μl of PBS solution of lethal toxin (100 μg PA+100 μg LF). In all experiments the rate of breathing was recorded every 10 minutes during the first 3 hours following injection, and animals were observed for survival for 7 days.

Treatment of spore-challenged mice. Mice used in all experiments were maintained under proper conditions with a 12-hour light/dark cycle in accordance with IACUC standards in the animal facility of the Biocon, Inc. (Rockville, Md.). Mice received food and water ad libitum. Groups of ten mice were randomly assigned for challenge and were observed for survival and signs of disease. The animals were inoculated i.p. by 1×10⁷ spores per mouse of Sterne strain. Treatment with ciprofloxacin (50 mg/kg, i.p.), rabbit sera (5 or 25 mg/kg, i.p.), or their combination once a day started 24 hours post spore challenge and continued for ten days. In all experiments, the animals were monitored for survival for at least 12 days after termination of treatment.

In vitro proteolytic activity of culture supernatant. Fluorescently labeled gelatin and casein were used as convenient substrates for testing the BACS proteolytic activity. The hydrolysis was inhibited by chemical inhibitors (phenanthroline and phosphoramidon), as well as the antisera against thermolysin-like enzymes and collagenases.

Statistical analysis. Kaplain-Meier open-end survival analysis was performed to compare results between treatment groups. Statistical significance was established as P<0.05 using log-rank test.

### EXAMPLE 2

Genomic Analysis of B. Anthracis Secreted Proteins as Potential Virulence Factors

In order to evaluate the pathogenic effect of B. anthracis proteins other than the known lethal and edema toxins, a nontoxigenic and nonencapsulated strain of B. anthracis, delta Ames, was analyzed. The delta Ames strain lacks both plasmids, pXO1 and pXO2.

First, an analysis of the chromosome sequence of the B. anthracis Ames strain was performed based on shared sequence homology with pathogenic factors in other bacterial species. (Supran et al., 2002; Read et al., 2003) This analysis revealed a variety of potential virulence-enhancing factors, including collagenases, phospholipases, haemolysins, proteases, and other enterotoxins. In fact, the B. cereus group of bacteria, which are pathogenic to humans or insects and includes B. anthracis, B. thuringiensis, and B. cereus, has more sequences that are predicted to be secreted proteins than does nonpathogenic B. subtilis (Read et al., 2003). These B. cereus group-specific genes represent the adaptations to a pathogenic lifestyle by the common ancestor, which was quite similar to B. cereus.

Most interesting of the secreted proteins is the group of proteases encoded on the B. anthracis chromosome that are shared in common with B. cereus, but are absent or relatively rare in the genomes of nonpathogenic bacteria. A large number of these proteases fall into clan MA [classified according to the MEROPS system, Barrett A J, 2004]. This clan includes thermolysin-like enzymes of the M4 family and others. The metallo-proteases (MPs) from several bacterial species belonging to this family are capable of causing massive internal hemorrhages and other death-threatening pathologies (Supuran et al., 2002; Sakata et al., 1996; Shin et al., 1996; Miyoshi et al., 1998; Okamoto et al., 1997).

Eleven protease families are present in B. anthracis and B. cereus, but absent in B. subtilis. Six of these eleven subfamilies encode MPs. Three of the MP subfamilies, namely the M6, M9B, and M20C subfamilies, are encoded on the bacterial chromosomes. Members of the M6 peptidase family are usually described as “immune inhibitors” because in B. thuringiensis they can inhibit the insect antibacterial response (Lovgren et al., 1990). The M20C peptidase subfamily represents exopeptidases (Biagini et al., 2001) that are an unlikely cause of tissue destruction or internal bleeding. But, the collagenolytic proteases of the M9B family have potential pathogenic functions.

This genomic analysis indicated that the M4 family of thermolysin/elastase-like neutral proteases and the M9 family of collagenases are virulence-enhancing factors of B. anthracis Ames strain.

### EXAMPLE 3

Hemorrhagic and Collagenolytic Activities of Anthrax Proteases

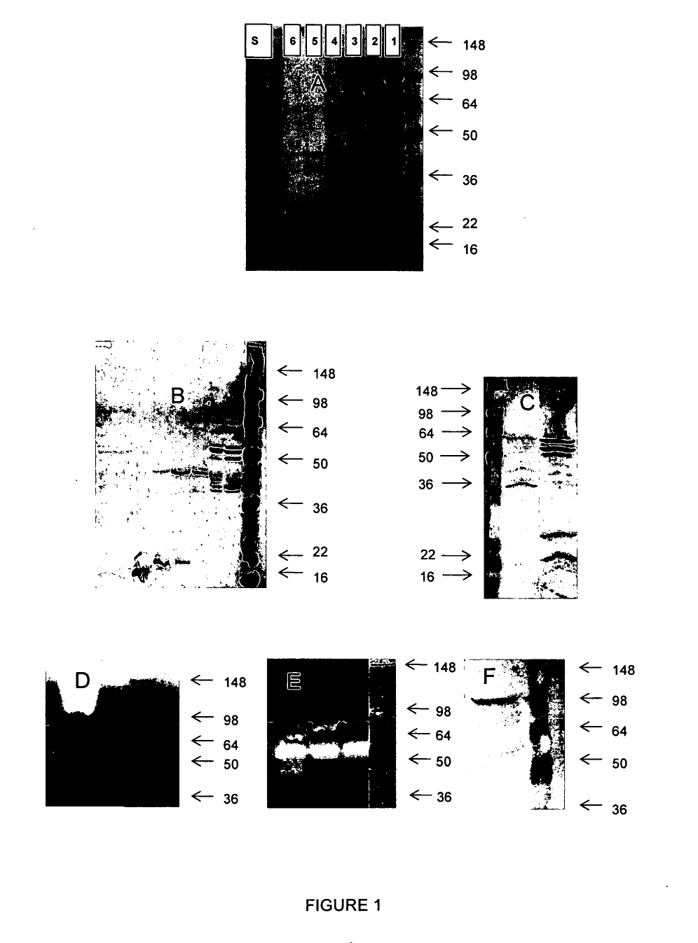

The proteins secreted by three Bacillus species (B. anthracis, B. cereus, and B. subtilis) into culture media were prepared by successively inoculating culture media with spores and incubating them overnight at 37° C. The bacterial cells were removed by centrifugation and the supernatant was sterilized by filtering through a 0.22μ filter. The supernatant was then concentrated 50-fold using an ultrafiltration device, such as an Amicon Ultra 15 filter (Millipore, MA) with a 10 KDa cutoff size. SDS-PAGE gel separation of culture supernatant (“BACS”) (FIG. 1A) demonstrates its protein content. Similar procedures were used to prepare culture supernatants for B. cereus (“BCCS”), ATCC #11778, and B. subtilis (“BSCS”), ATCC #23857.

Gelatinase and collagenase activity of BACS is readily detected by zymography using collagen type I or gelatin (denatured collagen) (FIGS. 1D and 1E). A major band of gelatinase activity corresponds to molecular mass of about 100 KDa, whereas a collagenase activity is represented by about 55 KDa proteins.

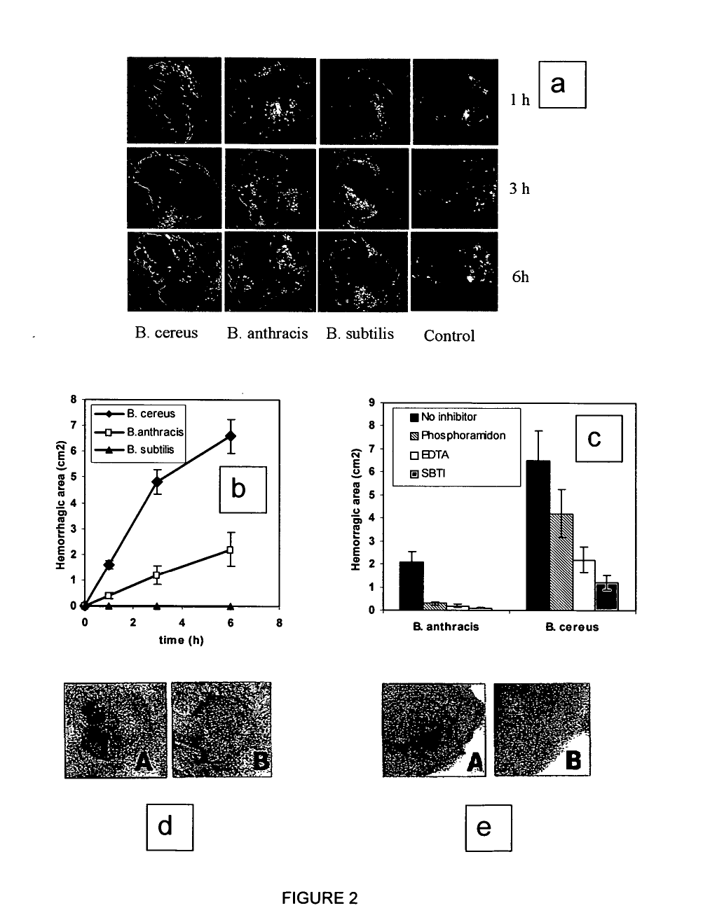

Next, the concentrated culture supernatants were tested in mice. Upon subcutaneous administration, mice developed hemorrhages of different intensity within several hours in response to the supernatants (FIGS. 2a and 2b). BCCS showed the highest activity followed by BACS, while BSCS was completely inactive. Therefore, B. anthracis and B. cerus secrete proteins with hemorrhagic effects.

In order to further confirm that there are other virulence factors involved in pathologic changes typical of anthrax infection, another experiment was conducted. The toxin and plasmid-free Ames strain of B. anthracis (delta-pX01/pX02) was cultured in LB media overnight. A cell-free supernatant was prepared. The purified supernatant was injected in the femoral artery region or the brain (via the carotid artery) of DBA-2 mice in order to induce pathologic changes described in anthrax patients.

Subcutaneous infection in the femoral artery region showed the development of hemorrhagic reaction in response to B. anthracis supernatants that did not contain toxin (FIG. 2d, panel A). No hemorrhages occurred after the injection of B. subtilis supernatant and LeTx (FIG. 2d, panel B).

In another experiment, a catheter was implanted into the right carotid artery towards the brain. The supernatant was infused with 1.5 pl/min flows for one hour. Severe extravasated red blood cell infiltration was observed in the animals infused with secreted proteins of B. anthracis (FIG. 2e, panel A), but not LeTx (FIG. 2e, panel B). These results correlated with 80% mortality in the group with secreted protein vs. 0% mortality in the LeTx group.

To more precisely define the proteins responsible for these hemorrhagic effects, chemical protease inhibitors were used. The inhibitors include phosphoramidon, which is a potent chelating inhibitor of thermolysin and other M4 bacterial metallo-endopeptidases (Komiyama et al., 1975), EDTA, which is specific for a broad range of MPs, and soybean trypsin inhibitor (“SBTI”), which is a reversible competitive inhibitor of trypsin and other trypsin-like proteases such as chymotrypsin, plasmin, and plasma kallikrein. Each of these chemical inhibitors effectively abrogated the hemorrhagic affect of BACS. (FIG. 2c).

Additional control experiments demonstrated that under the conditions of our test the hemorrhagic activity of thermolysin from B. thermophilicus was detectable in a dose range from 10 to 100 μg, similar to that for BACS (data not shown). While the inhibitors were almost completely effective against BACS, they displayed only partial protection against BCCS (FIG. 2c).

In addition, the murine serum raised against the recombinant protein corresponding to the mature form of the M4-type thermolysin-like neutral protease of B. anthracis (BA 3442) was also effective in suppressing the hemorrhagic effects of BACS and BCCS administered subcutaneously to mice (data not shown). In contrast, negative control experiments show that neither naive murine serum nor three irrelevant murine sera against B. anthracis hemolysins O, A and B (Klichko et al., 2003) showed anti-hemorrhagic activity (data not shown).

Overall, these results indicated that a hemorrhagic activity in BACS was represented by a single or several enzymes of the MP-type, while BSCS contained a more heterogeneous array of activities. This conclusion is consistent with the experimental data that B. anthracis possesses less extracellular proteolytic activity under standard laboratory conditions compared to B. cereus (Bonventre et al., 1963; Ezepchuk et al., 1969).

### EXAMPLE 4

Generation of Antibodies Against B. Anthracis MPs.

Because the composition of proteins in BACS is very complex, methods to detect and inhibit its components were developed. Several immune sera were raised in mice and rabbits using the antigens listed in Table 1 and used in Western blots of BACS proteins. The proteins of BACS were separated on an SDS-PAGE gel and subsequently transferred to a nitrocellulose membrane. The resulting blots were of low intensity, indicating proteolytic degradation during electrophoresis (FIG. 1A, left lane).

To avoid degradation, BACS was fractionated according to the molecular masses of its components on the Superdex® size exclusion column in the presence of EDTA as a chelating agent. Analysis of the column fractions in SDS-PAGE showed a complex pattern proteins bands (FIG. 1). Multiple proteins with a broad spectrum of molecular masses seem to be highly associated and migrate through the column as high molecular mass complexes. Several of these bands represent precursor and mature forms of proteins that result from specific proteolysis during the maturation process. In addition, there are unspecific proteolysis products, which can potentially contribute to the complexity of the composition.

Western blot experiments with column fractions revealed several discrete bands recognized by antibodies (FIG. 1). M4 proteases are represented by several bands at about 50 KDa, as well as by the bands at about 40 and 20 KDa. These bands likely correspond to different maturation forms of M4 proteases, for example enzymes lacking signal peptides and mature enzyme forms.

M9 collagenases are detected as a band with a molecular mass of about 98 kDa, which is close to the estimated mass of the pro-enzymes, however the major enzymatic activity corresponds to the 55 kDa size of the mature forms.

### EXAMPLE 5

In Vitro Proteolytic Activity of Culture Supernatant.

Caseinolytic and gelatinolytic activities of BACS are depicted in FIGS. 1D and E. The hydrolysis was inhibited by specific antibodies against thermolysin-like enzymes and collagenases.

### EXAMPLE 6

Acute Toxicity of B. Anthracis Culture Supernatants.

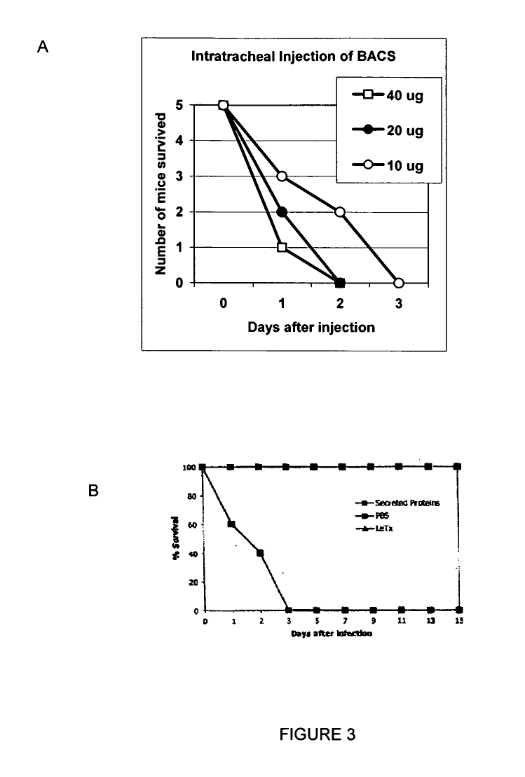

Although bacterial proteases are well known pathogenic factors, little information is available regarding their acute toxicity. To study their acute effects, BACS was introduced into mice by intratracheal administration to their lungs. This route models hemorrhagic mediastinitis and lung edema, which typically precede lethal outcome in late anthrax, with and lung damage considered to be a probable death-causing factor. In the experiment, mice were given different doses of BACS (10 μg to 40 μg of total protein) and were observed daily for lethality. FIG. 3A shows that depending on the dose, all mice died within 2 to 3 days, while the highest dose caused 80% mortality on the first day.

For histopathological examination, mice were given 100 μg of BACS protein, causing all of the animals to die within 3 to 4 hours. Postmortem harvested lungs revealed minimally or moderately severe focal intraalveolar acute hemorrhage, with no endothelial cell damage or vasculitis, and mild patchy congestion of medium-size blood vessels. There was evidence of focal platelet accumulation located within areas of hemorrhage or within vessels. In contrast, lethal toxin at a comparable dose (100 μg LF, 100 μg PA) caused neither mortality nor hemorrhage.

As seen in FIG. 3B, there was a rapid drop in the mortality curve in the group of animals receiving secreted proteins of B. anthracis, confirming the lethal activity of this substance. No animals infused with LeTx and PBS (used as controls) were lost. The condition of the mice that survived was identical to the intact, healthy animals.

These results show that factors other than LeTx and EdTx play a role in anthrax infection and that administration of BACS provides a better model of the acute toxic stages of anthrax disease than does lethal toxin.

### EXAMPLE 7

Protection of Mice Against Anthrax Using Protease Inhibitors.

Because chemical protease inhibitors effectively suppressed the proteolytic and hemorrhagic activity of BACS, their use as protective agents against B. anthracis infection was examined. Previously, successful application of an adjunct therapy against anthrax infection targeting both bacterial multiplication and host response to infection with a combination of antibiotic with caspase inhibitors has been reported by the inventors (Popov et al., 2004).

However, caspases act through an entirely different mechanism than proteases. Caspases are cysteine proteases that mediate cell apoptosis (cell death). Anthrax lethal toxin activates caspases in the host, which causes the cells to undergo programmed cell death. Using caspase inhibitors, inventors were targeting not a protein produced by anthrax, but a protein produced by humans.

Similarly to caspases, secreted MPs are not expected by those skilled in the art to directly interfere with bacterial multiplication and, thus, are not expected to be useful in a treatment of anthrax infection. To prove otherwise, inventors carried out a combination therapy experiment, in which antibiotic administration was complemented by protease inhibitor administration to target both bacterial and proteolytic factors.

In addition, efficacy of delayed treatment, which is initiated after a certain period of time following spore challenge, was investigated. Delayed treatment is desirable because patients often seek medical attention only after symptoms appear and treatment begins only after exposure has been confirmed. There is a particular need for delayed treatment because when administration of ciprofloxacin, the current antianthrax therapy, is delayed in mice only partial protective is achieved (Popov et al., 2004). Therefore, combination therapy comprising antibiotics and protease inhibitors were studied to determine if delayed treatment were feasible and whether a synergistic enhancement in survival could be obtained.

Two chemical inhibitors were chosen for the study of combination therapies. The first inhibitor, phosphoramidon, is a potent inhibitor of thermolysin and other bacterial metallo-endopeptidases, but not trypsin, papain, chymotrypsin or pepsin. This inhibitor only weakly inhibits collagenase. Phosphoramidon was found to be effective in suppressing the hemorrhagic effect of BACS. The second inhibitor, o-phenanthroline (1,10-phenanthroline), is a potent chelating inhibitor of M4 MPs, such as pseudolysin, as well as matrix MPs (Supuran et al., 2002).

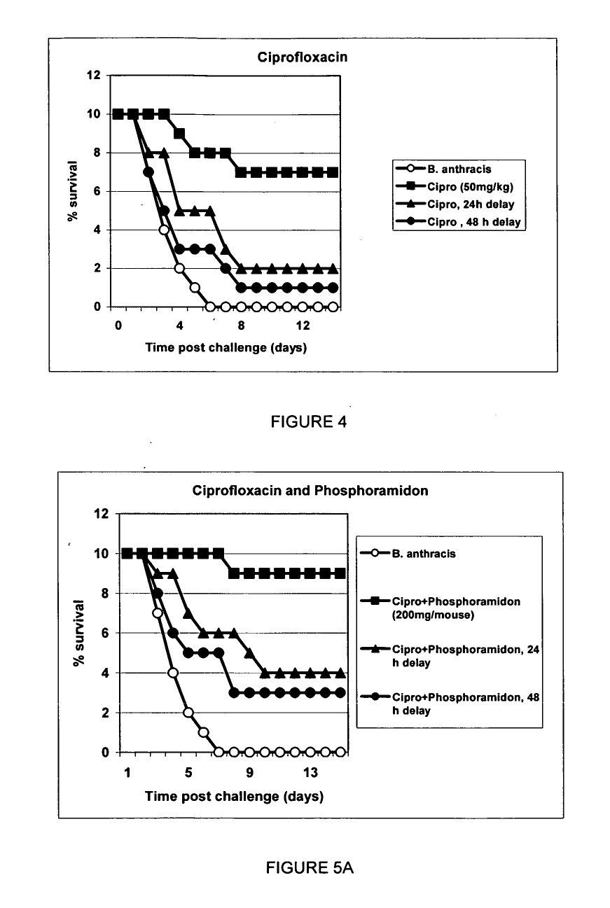

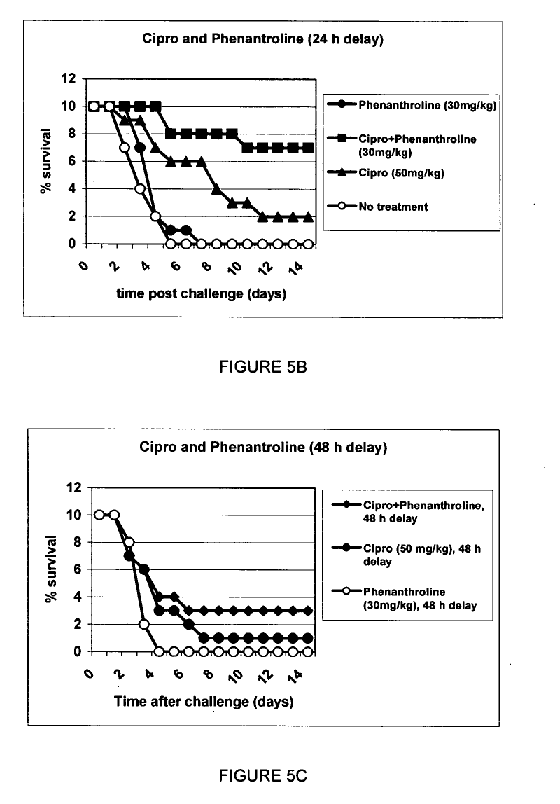

The results of three independent experiments of the combination therapy are presented in FIGS. 4 and 5. Mice were challenged intraperitoneally (ip) with about 1×10⁷ of B. anthracis Sterne spores. First, antibiotics alone, were examined (FIGS. 4 and 5A). Treatment with a single daily dose of ciprofloxacin (50 mg/kg, ip) began immediately after challenge, as well as at 24 hours or 48 hours post challenge, and continued for 10 days. Ciprofloxacin treatment initiated immediately after spore challenge was only 70% effective. While survival rate after a 24 hour delay declined sharply to 20%, although it remained statistically reliable (compared to untreated group, p=0.015). After a 48 hour delay, though, the antibiotic was ineffective (p=0.23) (FIG. 4).

Treatment with inhibitor in the absence of antibiotic did not increase survival, however the combination of ciprofloxacin with inhibitors displayed a dramatic increase in protection, especially when the inhibitor was o-phenanthroline. (FIGS. 5A-5C) Treatment with phenanthroline and ciprofloxacin, which was delayed by 24 hours protected 70% of animals, whereas only 20% survived when treated with ciprofloxacin alone (p=0.03 for these groups). When treatment was delayed 48 hours, there was a statistically reliable 30% increase in protection (relative to untreated spore-challenged group, p<0.05) in comparison to similar treatment with ciprofloxacin alone (relative to untreated spore-challenged group, p=0.23). (FIGS. 5B and 5C)

The combination of phosphoramidon and ciprofloxacin compared to ciprofloxacin alone also increased protection (FIG. 5A), however the observed differences are less statistically significant (p>0.05).

### EXAMPLE 8

Protection of Mice Against B. Anthracis Using Anti-Protease Sera

The inventors also tested an ability of antibodies raised against B. anthracis MPs to neutralize protease activity in vitro and in vivo. As in the experiments of Example 7 using inhibitors, mice were challenged intraperitoneally (ip) with about 30 LD50 of B. anthracis Sterne spores. Treatment with a single daily dose of ciprofloxacin (50 mg/kg, ip) began at 24-hours post challenge and was continued for 10 days. Immune sera was administered at a concentration of 25 mg/ml (ip) once daily.

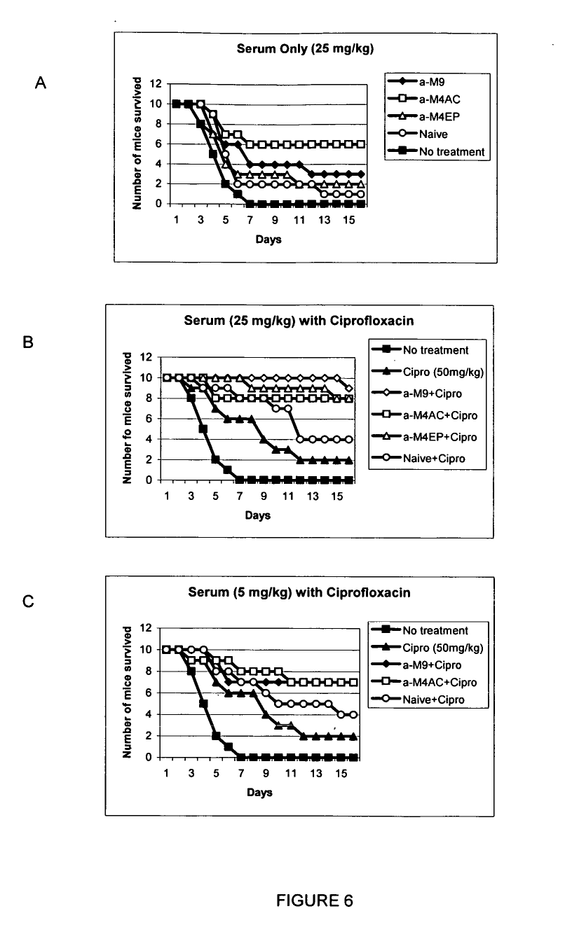

The immune sera displayed substantial differences in protective effects. Anti-M4 serum, M4AC, raised against the epitope(s) of the active center displayed the highest protection (60%), while the anti-collagenase serum (a-M9Coll) protected 30% of the mice. Anti-M4EP serum behaved similarly to naive serum. (FIG. 6A) Both a-M9Coll and a-M4EP sera demonstrated no statistically reliable difference in survival, compared to untreated mice (10%, p>0.05).

Combination treatment with both antibiotic and all studied immune sera, administered at the same dose (25 mg/kg) resulted in a synergistic effect and protected from 80 to 100% of the mice. (FIG. 6B) A lower serum dose (5 mg/kg) showed similar pattern of protection, however the effect of combination treatment was reduced to 70%. (FIG. 6C)

### EXAMPLE 9

Protection of Mice against Anthrax Using Additional Protease Inhibitors

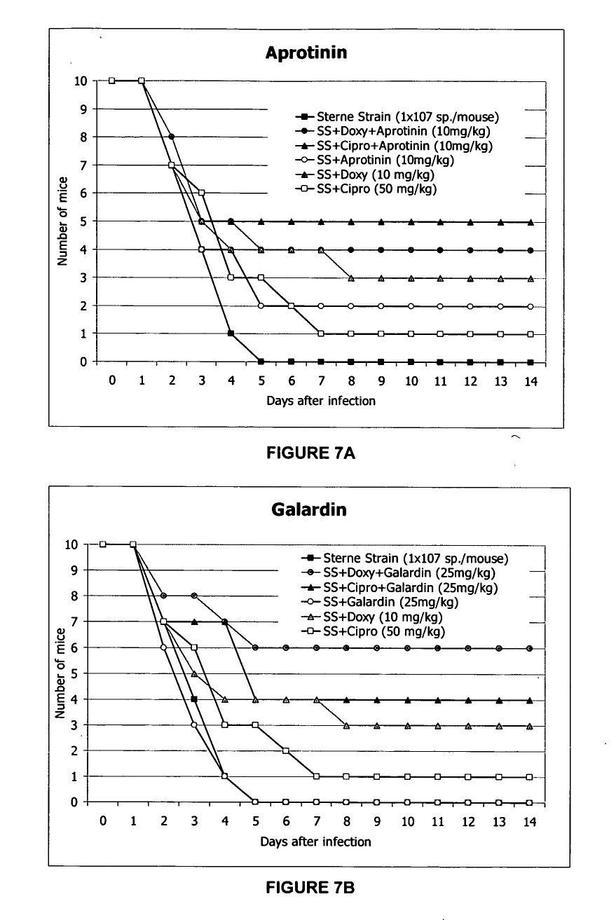

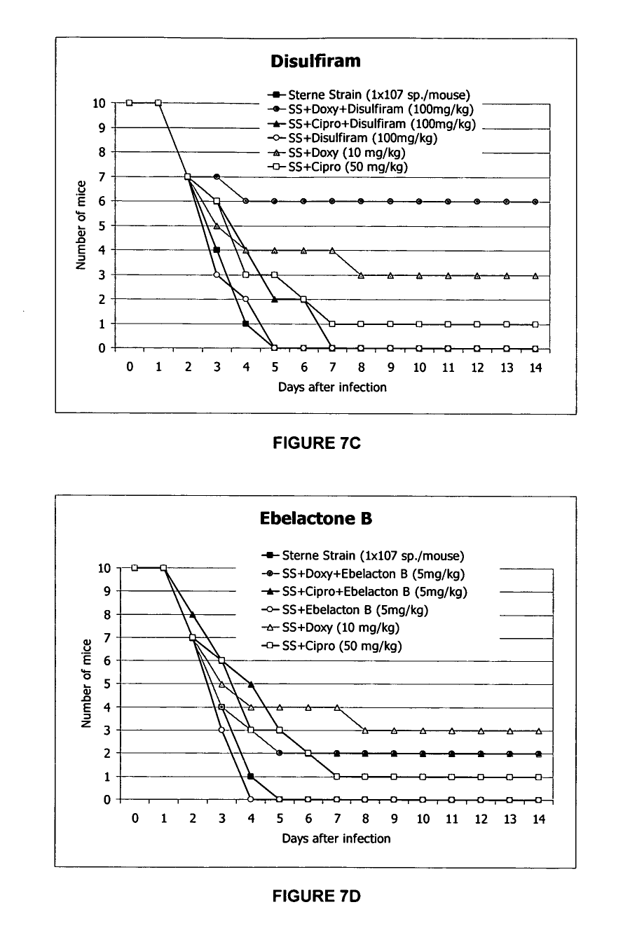

In addition, the efficacy of combination treatment using different antibiotics and inhibitors was studied. The inhibitors studied were chosen among the FDA approved drugs or the drugs already tested in clinical trials for other purposes. The results are presented in FIG. 7. There are varying degrees of protection demonstrating that the general approach of a combinational anthrax therapy with B. anthracis MP inhibitor and an antimicrobial agent, such as an antibiotic, is generally valid. Based on the description of this general approach and the specific examples that follow, those skilled in the art will be able to select the optimal inhibitor drug for a particular antimicrobial agent and to optimize the ratio of the inhibitor to the antimicrobial agent.

The experiments were conducted as follows. Mice were challenged intraperitoneally (i.p.) with approximately 1.5×10⁷ of B. anthracis spores. Treatment with a single daily dose of ciprofloxacin (50 mg/kg, i.p.) or doxycycline (10 mg/kg, i.p.), protease inhibitor, ciprofloxacin/protease, inhibitor, or doxycycline/protease inhibitor combination began either 24 (only ciprofloxacin/galardin-FIG. 7) or 48 hours post challenge and continued for 10 days. Under these conditions, the ciprofloxacin treatment was only 20% effective (in both cases-24 and 48 hours after infection) and doxycycline treatment only 30% effective.

Inhibitor treatment without antibiotic did not increase survival, however the combination of ciprofloxacin or doxcycline with inhibitors displayed a synergistic increase in protection. The 24 hour-delayed ciprofloxacin/galardin treatment increased survival up to 70% comparing to 20% survival in case of ciprofloxacin treatment alone (FIG. 7B). The 48 hour-delayed doxycycline/disulfuram (FIG. 7C) and doxycyclin/galardin (FIG. 7B) treatments protected 60% of the animals, whereas there was only 30% survival in the group with doxycycline alone. The 48 hour-delayed ciprofloxacin/aprotinin treatment protected 50% of the animals (FIG. 7A). Finally, ebelactone B did not increase survival over the effects of doxycycline alone (FIG. 7D).

The present invention may be embodied in other specific forms without departing from its essential characteristics. The described embodiments are to be considered in all respects only as illustrative and not as restrictive. The scope of the invention is, therefore, indicated by the appended claims rather than by the foregoing description. All changes which come within the meaning and range of the equivalence of the claims are to be embraced within their scope.

### EXAMPLE 10

Efficacy Study in Rabbit Model

The rabbits (New Zealand white, 2-3.5 kg) are challenged with an aerosol of B. anthraces Ames spores. [Pitt et al. 2001, Fellows et al, 2001]. The spores are prepared by dilution in sterile water to a concentration of 2.2-2.8×109 CFUIml, then are heatshocked at 60° C. for 45 minutes, and divided into 8 ml aliquots. Respiratory minute volumes are measured by whole body plethysmography prior to challenge. The rabbits are exposed to the spore aerosol (generated by a jet collision nebulizer) into the nose. The rabbits are split into different groups with 10 rabbits in each group. The rabbits receive the spore concentration equivalent to the amount needed to cause inhalational anthrax in humans.

Treatment is conducted using the best two combinations chosen from the mouse experiments and are initiated from mid- to advanced stage of the anthrax infection course. The therapy's parameters, antibiotic and protease inhibitors concentrations are determined in the foregoing mouse experiments and recalculated for a rabbit model. In addition to the evaluation of survival, rabbits are examined for bacteremia by drawing 0.2 ml of blood for at least 14 days after termination of treatment and plating on dishes with brain-heart agar. The therapeutic efficacy is determined in comparison with a single antibiotic therapy using ciprofloxacin or doxycycline.

### EXAMPLE 11

Pharmacokinetics

Pharmacokinetics (PK) studies are preformed for protease inhibitors and protease inhibitor/antibiotic combination in compliance with the FDA requirements for new drug development. In assessment of the PK characteristics, 5 different doses (5, 10, 25, 50, 100 mg/kg per body weight) are tested and 10 mice are used fore each dose. Blood samples are collected from each drug-treated mouse through orbital bleeding.

At minimum, blood samples are collected to determine plasma drug concentrations at the approximate time of maximum concentration (peak or Cmax) and at the end of the dosing interval (trough or Cmin), after first dose administration and for several successive days after steady state has been attained (at least 5 Cmax and 5 Cmin determinations). The concentration of the protease inhibitors in the mouse serum is determined by using radioimmuoassay and/or HPLC assay as adapted by most pharmaceutical manufacturers and approved by the FDA.

### EXAMPLE 12

Study of Acute and Sub-Acute Toxicity of The Inhibitors Alone and in Combination with Antibiotics

Protease inhibitors and protease inhibitor/antibiotic combinations are examined for acute and sub-acute toxicity using a mouse model. In the acute toxicity study, intraperitoneal injection is used to administer the protease inhibitors (100 mg/kg body weight) alone or in combination with the antibiotics in one or more doses during a period not exceeding 24 hours.

Subsequently, the animal is observed up to 14 days after pharmaceutical administration (Guidance for Industry Single Dose Acute Toxicity Testing for Pharmaceuticals Center for Drug Evaluation and Research (CDER) August 1996). All mortalities, clinical signs, time of onset, duration, and reversibility of toxicity are recorded. Gross necropsies is performed on all animals, including those sacrificed moribund, found dead, or terminated at 14 days. Pathology and histopathology of selected tissues and organs such as brain, lungs, liver, and spleen are monitored at an early time and at termination.

The sub-acute toxicity is carried out by using 28-day repeated dose tests. The study provides information on the major toxic effects, indicates target organs and the possibility of accumulation, and provides an estimate of a no-observed-adverse-effect level of exposure, which can be used in selecting dose levels for chronic studies and for establishing safety criteria for human exposure.

The test substances (protease inhibitors alone and in combination with antibiotics) are intraperitoneally administered daily in graduated three doses (25, 50, 100 mg/kg body weight) to several groups of experimental animals, one dose level per group, for a period of 28 days. At least 10 animals (five female and five male) are used at each dose level. During the administration, the animals are observed closely for signs of toxicity. Observations include, but are not limited to, changes in body weight, skin, fur, eyes, mucous membranes, occurrence of secretions and excretions, and autonomic activity (e.g., lacrimation, pilo-erection, pupil size, unusual respiratory pattern). Changes in gait, posture, and response to handling as well as the presence of clonic or tonic movements, stereotypes (e.g., excessive grooming, repetitive circling), or any unusual behavior are also recorded.

Animals, which die or are killed during the test are necropsied. At the conclusion of the test, surviving animals are also killed and necropsied. Pathology and histopathology of selected tissues and organs such as brain, lungs, liver, and spleen are monitored at termination. Blood cell count and chemical profile is examined on day 5, 10, 15 after administration of the drug using samples collected through orbital bleeding.

### EXAMPLE 13

Development of a Combined (“All-In-One”) Therapeutic Preparation

Based on the results obtained from the animals, two best combinations of antibiotics with the protease inhibitors are selected for the development of a combined (“all-in-one”) therapeutic preparation. Optimal molecular ratios of an antibiotic and a protease inhibitor are developed by testing the efficacy of various combinations in the murine model.

In one experiment, the antibiotic concentration is maintained at one level while the concentration of the protease inhibitor is varied to make a variety of preparations and test them in murine anthrax model as described above. The best combination is selected after at least three trials by two independent laboratories.

A comparison of the combined preparation's therapeutic efficacy, pharmacokinetics, and toxicity with the regimen of a single agent administration of either antibiotics or protease inhibitors is performed. The same dosage of a combined preparation or a single agent are administered in parallel to determine the synergetic therapeutic efficacy and effects on pharmacokinetics and toxicity in murine and rabbit models as described above.

Stability of the preparation at room temperature and in refrigerated conditions for the period of 1, 3, and 6 months is tested. The concentration of the drugs in the preparation is determined using radioimmunoassay and/or 1-IPLC chromatography.

The composition of the combined preparation is optimized for a small-scale production. Effects of a range of factors, including, but not limited to, temperature, type of diluent and its concentration, transportation, and storage conditions, on the composition are studied.

A predetermined number of therapeutic doses of the composition is prepared for and used in pre-clinical and clinical studies.

### REFERENCES

The following references are cited herein. The entire disclosure of each reference is relied upon and incorporated by reference herein.
1. Barrett A J. Bioinformatics of proteases in the MEROPS database. Curr Opin Drug Discov Devel. 2004; 7(3):334-41.
2. Biagini A and Puigserver A. Sequence analysis of the aminoacylase-1 family. A new proposed signature for metalloexopeptidases. Comp Biochem Physiol B Biochem Mol. Biol. 2001; 128(3):469-81.
3. Bonventre P F and Eckert N J. The biologic activities of Bacillus anthracis and Bacillus cereus Culture Filtrates. Am J. Pathol. 1963 August; 43:201-11.
4. Cui X, et al. Lethality during continuous anthrax lethal toxin infusion is associated with circulatory shock but not inflammatory cytokine or nitric oxide release in rats. Am J Physiol Regul Integr Comp Physiol. 2004; 286(4):R699-709.
5. Ezepchuk IuV, et al. [On the toxic and various biochemical properties of filtrates of B. anthracis and cereus] Zh Mikrobiol Epidemiol Immunobiol. 1969; 46(6):19-23 [Article in Russian].
6. Fellows, P. F. et al., Efficacy of a human anthrax vaccine in guinea pigs, rabbits, and rhesus macaques against challenge by Bacillus anthracis isolates of diverse geographical origin. Vaccine 19, 2001; 3241-3247.
7. Inglesby T V, et al. Working Group on Civilian Biodefense. JAMA. Anthrax as a biological weapon, 2002: updated recommendations for management. 2002; 287(17):2236-52.
8. Ivanova N, et al. Genome sequence of Bacillus cereus and comparative analysis with Bacillus anthracis. Nature. 2003; 423(6935):87-91.
9. Klein F, et al. Pathophysiology of anthrax. J Infect Dis. 1966; 116(2):123-38.
10. Klichko V I, et al. Anaerobic induction of Bacillus anthracis hemolytic activity. Biochem Biophys Res Commun. 2003; 303(3):855-62.
11. Kunst F, et al. The complete genome sequence of the gram-positive bacterium Bacillus subtilis. Nature. 1997; 390(6657):249-56.
12. Lovgren A, et al. Molecular characterization of immune inhibitor A, a secreted virulence protease from Bacillus thuringiensis. Mol. Microbiol. 1990; 4(12):2137-46.
13. Miyoshi S, et al. Characterization of the hemorrhagic reaction caused by Vibrio vulnificus metalloprotease, a member of the thermolysin family. Infect Immun. 1998; 66(10):4851-5.
14. Okamoto T, et al. Activation of human matrix metalloproteinases by various bacterial proteinases. J Biol. Chem. 1997; 28; 272(9):6059-66.
15. Panchal, R G, et al., Identification of small molecule inhibitors of anthrax lethal factor. Nature Structural & Molecular Biology 2004, 11(1): 67-72.
16. Pitt M. L. M et al, In vitro correlate of immunity in a rabbit model of inhalational anthrax. Vaccine 19, 2002; 4768-4773.
17. Popov S G, et al. Effect of Bacillus anthracis lethal toxin on human peripheral blood mononuclear cells. FEBS Lett. 2002b; 527(1-3):211-5.
18. Popov S G, et al. Systemic cytokine response in murine anthrax. Cell Microbiol. 2004; 6(3): 225-33
19. Read T D, et al. The genome sequence of Bacillus anthracis Ames and comparison to closely related bacteria. Nature. 2003; 423(6935):81-6.
20. Read T D, et al. Comparative genome sequencing for discovery of novel polymorphisms in Bacillus anthracis. Science. 2002; 296(5575):2028-33.
21. Sakata Y, et al. Bradykinin generation triggered by Pseudomonas proteases facilitates invasion of the systemic circulation by Pseudomonas aeruginosa. Microbiol Immunol. 1996; 40(6):415-23.
22. Shin Y H, et al. Further evidence of bradykinin involvement in septic shock: reduction of kinin production in vivo and improved survival in rats by use of polymer tailored SBTI with longer t1/2. Immunopharmacology. 1996; 33(1-3):369-73.
23. Supuran C T, et al. Bacterial protease inhibitors. Med Res Rew. 2002; 22(4):329-72.

What is claimed is:

1. A composition for treating an anthrax infection comprising a therapeutically effective amount of at least one B. anthracis metalloprotease (MP) inhibitor, wherein MP is other than lethal factor (LF).

2. The composition of claim 1, wherein the MP is a member of M4 or M9 family of MPs.

3. The composition of claim 2, wherein the MP is encoded by the gene BA3442, BA0555, BA3299, BA3584, BA5282, A0599, or BA2730.

4. The composition of claim 1, wherein the MP inhibitor is a chemical inhibitor.

5. The composition of claim 4, wherein the chemical inhibitor is selected from the group consisting of ethylenediamine-tetraacetic acid (EDTA), phosphoramidon, soybean trypsin inhibitor (SBTI), o-phenanthroline, aprotinin, galardin, disulfram, and ebelactone B.

6. The composition of claim 1, wherein the MP inhibitor is an antibody raised against a MP.

7. The composition of claim 6, wherein the antibody is raised against at least one peptide comprising a sequence SEQ ID NO:1, HEFTHYLQGRYEVPGL; SEQ ID NO:2, DVIGHELTHAVTE; SEQ ID NO:3, ADYTRGQGIETY, or a conservative modification of any of these sequences.

8. The composition of claim 7, wherein the antibody is a polyclonal or a monoclonal antibody.

9. The composition of claim 1, wherein the MP inhibitor is an antiserum containing at least one antibody raised against at least one peptide comprising a sequence SEQ ID NO:1, HEFTHYLQGRYEVPGL; SEQ ID NO:2, DVIGHELTHAVTE; SEQ ID NO:3, ADYTRGQGIETY, or a conservative modification of any of these sequences.

10. The composition of claim 1, 4, or 6 further comprising a physiologically acceptable antimicrobial agent.

11. A composition for treating an anthrax infection comprising a therapeutically effective ratio of at least one B. anthracis MP inhibitor and an antimicrobial agent.

12. The composition of claim 11, wherein the MP inhibitor is a chemical inhibitor.

13. The composition of claim 12, wherein the chemical inhibitor is selected from the group consisting of EDTA, phosphoramidon, SBTI, o-phenanthroline, aprotinin, galardin, disulfram, and ebelactone B.

14. The composition of claim 11, wherein the MP inhibitor is an antibody raised against a MP.

15. The composition of claim 14, wherein the antibody is raised against at least one peptide comprising a sequence SEQ ID NO:1, HEFTHYLQGRYEVPGL; SEQ ID NO:2, DVIGHELTHAVTE; SEQ ID NO:3, ADYTRGQGIETY, or a conservative modification of any of these sequences.

16. The composition of claim 15, wherein the antibody is a monoclonal or a polyclonal antibody.

17. The composition of claim 11, wherein the antimicrobial agent is an antibiotic.

18. The composition of claim 17, wherein the antibiotic is selected from a group of antibiotics effective against anthrax infection.

19. The composition of claim 18, wherein the antibiotic is selected from a group consisting of fluoroqinalones, tetracyclines, and β lactams.

20. The composition of claim 19, wherein the antibiotic is ciprofloxacin hydrochloride (ciprofloxacin) or doxcycline.

21. The composition of claim 20, wherein the antibiotic is ciprofloxacin and the chemical inhibitor selected from a group consisting of o-phenanthroline, aprotinin, and galardin.

22. The composition of claim 20, wherein the antibiotic is doxycycline and the chemical inhibitor is disulfuram or galardin.

23. The composition of claim 11 further comprising at least one additional active ingredient effective against anthrax infection.

24. The composition of claim 11, wherein the antimicrobial agent and the B. anthracis MP inhibitor are administered at the same time.

25. The composition of claim 11, wherein the antimicrobial agent and the B. anthracis MP inhibitor are administered serially, with either the antibiotic or the MP inhibitor administered first.

26. A method for treating anthrax infection in a human or an animal subject comprising administering to the subject a therapeutically effective amount of a composition comprising at least one B. anthracis MP inhibitor.

27. The method of claim 26, wherein the MP is a member of M4 or M9 family of MPs.

28. The method of claim 26, wherein the MP inhibitor is a chemical inhibitor selected from the group consisting of EDTA, phosphoramidon, SBTI, o-phenanthroline, aprotinin, galardin, disulfram, and ebelactone B.

29. The method of claim 26, wherein the MP inhibitor is an antibody raised against a MP.

30. The method of claim 29, wherein the antibody is raised against at least one peptide comprising a sequence SEQ ID NO:1, HEFTHYLQGRYEVPGL; SEQ ID NO:2, DVIGHELTHAVTE; SEQ ID NO:3, ADYTRGQGIETY, or a conservative modification of any of these sequences.

31. A method for treating anthrax infection in a human or an animal subject, wherein the method comprises administering to the subject a composition comprising a therapeutically effective ratio of at least one B. anthracis MP inhibitor and an antimicrobial agent.

32. The method of claim 31, wherein the antimicrobial agent is an antibiotic selected from a group consisting of fluoroqinalones, tetracyclines, and B lactams.

33. The method of claim 32, wherein the antibiotic is ciprofloxacin or doxcycline.

34. The method of claim 26 or claim 31, wherein the administering step is delayed at least 24 hours from the time of exposure of the subject to B. anthracis.


SEQUENCE LISTING (3 sequences)

SEQ ID NO: 1 - protein, 16 aa, Artificial
  chemically synthesized peptide
HEFTHYLQGRYEVPGL

SEQ ID NO: 2 - protein, 13 aa, Artificial
  chemically synthesized peptide
DVIGHELTHAVTE

SEQ ID NO: 3 - protein, 12 aa, Artificial
  chemically synthesized peptide
ADYTRGQGIETY